(19) United States
(12) Patent Application Publication
(10) Pub. No.: US 2026/0183083 A1
(43) Pub. Date: Jul. 2, 2026

(54) Q-FACTOR CONTROL FOR USE WITH RETAINED SURGICAL ITEM DETECTION SYSTEM

(71) Applicant: Covidien LP, Mansfield, MA (US)

(72) Inventors: Edward L. Brannan, Erie, CO (US); Frank T. Smith, Clark, CO (US)

(21) Appl. No.: 18/863,870

(22) PCT Filed: May 12, 2023
(86) PCT No.: PCT/IB2023/054946

§ 371 Date: Nov. 7, 2024

Related U.S. Application Data
(60) Provisional application No. 63/342,685, filed on May 17, 2022.

(51) Int. Cl. A61B 90/98; H01Q 7/00; H04B 5/43; H04B 5/77

(52) U.S. Cl. CPC A61B 90/98; H01Q 7/005; H04B 5/77; H04B 5/43

(57) ABSTRACT

An interrogation and detection system for detection of surgical implements within a patient's body includes an RF tag configured to transmit a return signal when energized, a signal generator configured to generate an energizing signal for the RF tag, an antenna operably coupled to the signal generator and configured to receive the return signal transmitted by the RF tag, a processor, and a memory. The memory includes instructions stored thereon, which when executed by the processor, cause the system to receive the return signal, detect an impedance of the antenna based on the return signal, and determine a ratio of real impedance to reactive impedance based on the detected impedance.

### FIELD

This disclosure relates generally to interrogation and detection systems for radio-frequency (RF) tags, and more particularly, interrogation, detection and inventory systems for radio-frequency (RF) tags for use within surgical sites.

### BACKGROUND

It is often useful to determine whether objects associated with a surgery are present in a patient's body before completion of the surgery. Such objects may take a variety of forms. For example, the objects may take the form of instruments, for instance, scalpels, scissors, forceps, hemostats, and/or clamps. Also, for example, the objects may take the form of related accessories and/or disposable objects, for instance, surgical sponges, gauzes, and/or pads. Failure to locate an object before closing the patient may require additional surgery, and in some instances, may have unintended medical consequences.

Accordingly, there is a need for a technology that is capable of providing both presence detection and tagged surgical item/implement identification functionality in the medical setting, as well as inventory controls of the tagged items/implements. Specifically, detecting the presence of, identifying, and maintaining inventory of tagged surgical items and materials that are used during the execution of a medical procedure. Technologies exist that enable these functions both individually as well as in conjunction with each other, but the methods and packaging of the discrete solutions used are not ideal for the application. More specifically, the components attached or affixed to the items being tracked are either too large physically and present nuisances or obstacles in the execution of the procedure, or the detection and identification performance of the solution may degrade rapidly in the presence of variable and uncontrolled dielectric or conductive materials.

Accordingly, there are needs for improvements in presence detection, tagged item identification, and inventory functionality in the medical setting.

### SUMMARY

This disclosure relates to systems for detection of surgical objects and devices used in body cavities during surgery, specifically systems and methods for controlling the electrical damping factor (Q factor) of an antenna used in such systems.

In accordance with aspects of the disclosure an interrogation and detection system for detection of surgical implements within a patient's body is presented. The system includes an RF tag configured to transmit a return signal when energized, a signal generator configured to generate an energizing signal for the RF tag, an antenna operably coupled to the signal generator and configured to receive the return signal transmitted by the RF tag, a processor, and a memory. The memory includes instructions stored thereon, which when executed by the processor, cause the system to receive the return signal, detect an impedance of the antenna based on the return signal, the impedance including a real impedance and a reactive impedance, and determine a ratio of real impedance to reactive impedance based on the detected impedance.

In an aspect of the disclosure, the instructions when executed by the processor, may further cause the system to set the ratio to be a predetermined constant value based on the determined ratio of real impedance to reactive impedance.

In another aspect of the disclosure, the instructions when executed by the processor, may further cause the system to set the ratio to be within a predetermined value range based on the determined ratio of real impedance to reactive impedance.

In a further aspect of the disclosure, setting the ratio to the predetermined value may be performed by adjusting a real and/or a reactive element of the antenna.

In yet a further aspect of the disclosure, the impedance may be detected based on a current through the antenna.

In an aspect of the disclosure, the adjusting may be performed by changing the current through the antenna.

In another aspect of the disclosure, the adjusting may be performed by changing a tuning voltage to a voltage-controlled resistor.

In yet another aspect of the disclosure, the current may be measured by a root mean square (RMS) converter.

In a further aspect of the disclosure, the instructions when executed by the processor, may further cause the system to determine whether a return signal was received, from a RF tag that marks a surgical implement used in a procedure, via the antenna.

In an aspect of the disclosure, the instructions, when executed by the processor, may further cause the system to transmit information to a display configured to display information related to the RF tag.

In accordance with aspects of the disclosure, an interrogation and detection system for detection of surgical implements within a body of a patient is presented. The interrogation and detection system includes an RF tag configured to transmit a return signal when energized, a signal generator configured to generate an energizing signal for the RF tag, an antenna operably coupled to the signal generator and configured to receive the return signal transmitted by the RF tag, a processor, and a memory. The memory includes instructions stored thereon, which when executed by the processor, cause the system to: measure a current through the antenna, determine a Q factor of the antenna based on the measured current, and compare the determined Q factor to a predetermined value.

In yet a further aspect of the disclosure, the instructions when executed by the processor, may further cause the system to adjust the Q factor of the antenna based on the comparison.

In an aspect of the disclosure, the adjusting may be performed by changing the current through the antenna.

In another aspect of the disclosure, the adjusting may be performed by changing a tuning voltage to a voltage-controlled resistor.

In yet another aspect of the disclosure, the current may be measured by an RMS converter.

In an aspect of the disclosure, a computer-implemented method for detection of surgical implements within a body of a patient is presented. The method includes generating, by a signal generator, an energizing signal for an RF tag affixed to a surgical implement, receiving a return signal from the RF tag by an antenna operably coupled to the signal generator, measuring a current through the antenna, determining a Q factor of the antenna based on the measured current, comparing the determined Q factor to a predetermined value, and adjusting the Q factor of the antenna based on the comparison.

In another aspect of the disclosure, the adjusting may be performed by changing the current through the antenna.

In yet another aspect of the disclosure, the method may further include determining whether a return signal was received from the RF tag that marks a surgical implement used in a procedure via the antenna.

In a further aspect of the disclosure, the current may be measured by an RMS converter.

In an aspect of the disclosure, the method may further include transmitting information to a display configured to display information related to the RF tag.

### BRIEF DESCRIPTION OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged or shrunk and positioned to improve drawing legibility. Further, the particular shapes of the elements as drawn are not intended to convey any information regarding the actual shape of the particular elements and have been solely selected for ease of recognition in the drawings.

Various aspects of the presently disclosed antennae, RF tags, and articles containing them are described herein below with reference to the drawings.

FIG. 1 is a schematic diagram illustrating an interrogation and detection system configured for detecting an object within a patient that is tagged with an RF tag according to one illustrated aspect;

FIG. 2 is a functional block diagram of a controller configured for use with the interrogation and detection system of FIG. 1 in accordance with embodiments of the disclosure;

FIG. 3A is a schematic of a control loop for use with the interrogation and detection system of FIG. 1 in accordance with embodiments of the disclosure;

FIG. 3B is a schematic of a nested control loop for use with the interrogation and detection system of FIG. 1 in accordance with embodiments of the disclosure;

FIG. 4 is a schematic of an electrical damping factor control circuit of the control loop of FIG. 3A in accordance with embodiments of the disclosure;

FIG. 5 is a schematic of a loop filter of the control loop of FIG. 3A in accordance with embodiments of the disclosure;

FIG. 6 is flow diagram for a computer-implemented method for detection of surgical implements within a patient's body in active use within a surgical site; and

FIG. 7 is a graph of electrical damping factor vs frequency as a real component of a tuning circuit is increased.

### DETAILED DESCRIPTION

In the following description, certain specific details are set forth in order to provide a thorough understanding of disclosed aspects. However, one skilled in the relevant art will recognize that aspects may be practiced without one or more of these specific details or with other methods, components, materials, etc. In other instances, well-known structures associated with transmitters, receivers, or transceivers have not been shown or described in detail to avoid unnecessarily obscuring descriptions of the aspects.

Reference throughout this specification to “one aspect” or “an aspect” means that a particular feature, structure, or characteristic described in connection with the aspect is included in at least one aspect. Thus, the appearances of the phrases “in one aspect” or “in an aspect” in various places throughout this specification are not necessarily all referring to the same aspect. Furthermore, the particular features, structures, or characteristics may be combined in any suitable manner in one or more aspects.

In an interrogation and detection system, the electrical damping factor (Q factor) can significantly affect the matching network. A matching network is necessary to present an output impedance to the RF amplifier that matches the impedance of the input of the antenna so electrical reflections are minimized. If the Q factor of the load can be fixed, then the matching network can be greatly simplified or eliminated. As an antenna is waved over a patient, the dielectric constant of the tissue of the patient may change the Q factor and/or tuning of the antenna. Since the Q factor can be a variable quantity or value, the disclosure describes a circuit that controls the Q factor and enables consistency of power delivery to the antenna.

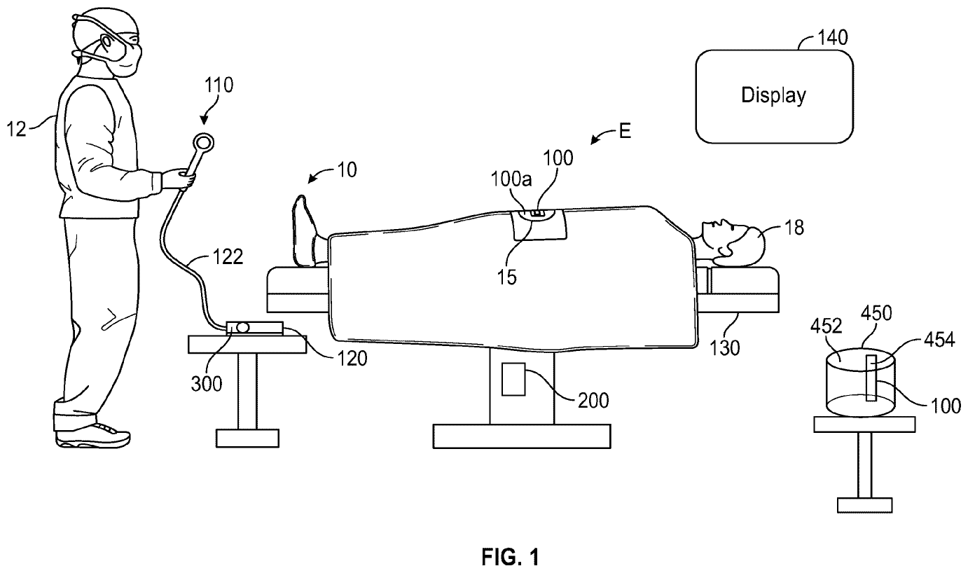

FIG. 1 depicts an interrogation and detection system 10 for detection of radio-frequency (RF) tags to ascertain the presence or absence of items, implements or objects 100a in a patient 18. The interrogation and detection system 10 generally includes a signal generator 300 and antenna 110 coupled to the signal generator 300 by one or more communication paths, for example, coaxial cable 122. In one aspect of the interrogation and detection system 10, the antenna 110 may be hand held or embedded in a mattress.

The object 100a may take a variety of forms, for example, instruments, accessories, and/or disposable objects useful in performing surgical procedures. For instance, the object 100a may take the form of scalpels, scissors, forceps, hemostats, and/or clamps. Also, for example, the objects 100a may take the form of surgical sponges, gauze, and/or padding. The object 100a is tagged, carrying, attached, or otherwise coupled to an RF tag 100. Aspects of the interrogation and detection system 10 disclosed herein are particularly suited to operate with one or more RF tags 100, which are not accurately tuned to a chosen or selected resonant frequency. Consequently, the RF tags 100 do not require high manufacturing tolerances or expensive materials and thus may be inexpensive to manufacture. The RF tag may include an inductor and capacitor that forms a resonant tank or may include an RFID tag that includes an inductor and a capacitor as well as the circuitry to enable an ID function.

In use, the medical provider 12 may use the detection system 10 in order to detect the presence or absence of the one or more RF tags 100 and hence an object 100a in the patient 18. For a detailed description of an exemplary interrogation and detection system, reference may be made to commonly owned U.S. Patent Application Publication No. 2004/0250819 to Blair et al., titled “Apparatus and Method for Detecting Objects Using Tags and Wideband Detection Device,” filed Mar. 29, 2004, the entire contents of which are hereby incorporated by reference herein.

In aspects, the signal generator 300 may generate an energizing signal for RF tag 100. The RF tag 100 may be affixed to a surgical implement. An antenna 110 may receive a return signal from the RF tag 100. The controller 200 may determine whether a return signal was received from an RF tag 100 that marks a surgical implement used in a procedure via the antenna 110. Then the controller may transmit information to a display 140 to display information related to the RF tag 100.

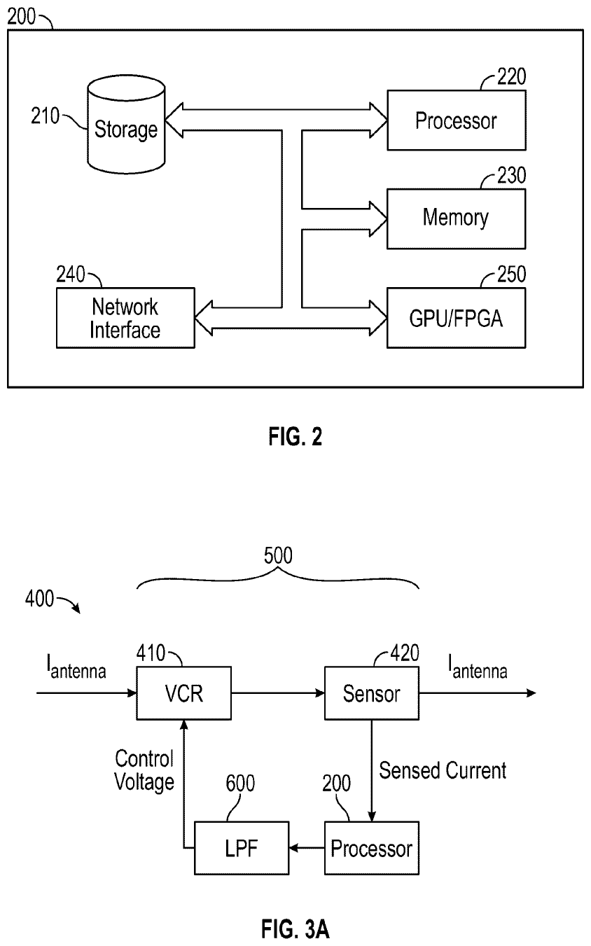

Now referring to FIG. 2, controller 200 includes a processor 220 connected to a computer-readable storage medium or a memory 230. The computer-readable storage medium or memory 230 may be a volatile type of memory, e.g., RAM, or a non-volatile type of memory, e.g., flash media, disk media, etc. In various aspects of the disclosure, the processor 220 may be another type of processor such as a digital signal processor, a microprocessor, an ASIC, a graphics processing unit (GPU), a field-programmable gate array (FPGA), or a central processing unit (CPU). In certain aspects of the disclosure, network inference may also be accomplished in systems that have weights implemented as memristors, chemically, or other inference calculations, as opposed to processors.

In aspects of the disclosure, the memory 230 can be random access memory, read-only memory, magnetic disk memory, solid-state memory, optical disc memory, and/or another type of memory. In some aspects of the disclosure, the memory 230 can be separate from the controller 200 and can communicate with the processor 220 through communication buses of a circuit board and/or through communication cables such as serial ATA cables or other types of cables. The memory 230 includes computer-readable instructions that are executable by the processor 220 to operate the controller 200. In other aspects of the disclosure, the controller 200 may include a network interface 240 to communicate with other computers or to a server. A storage device 210 may be used for storing data. The disclosed method may run on the controller 200 or on a user device, including, for example, on a mobile device, an IoT (“internet of things”) device, or a server system.

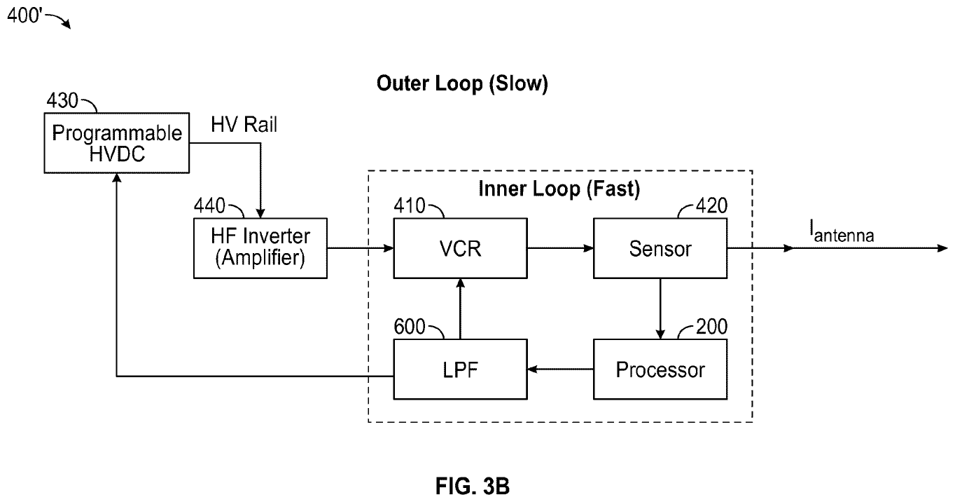

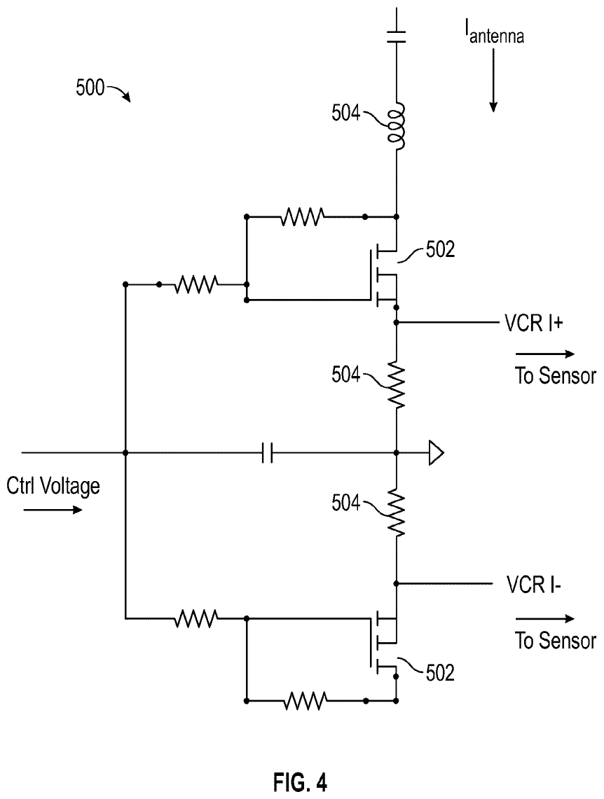

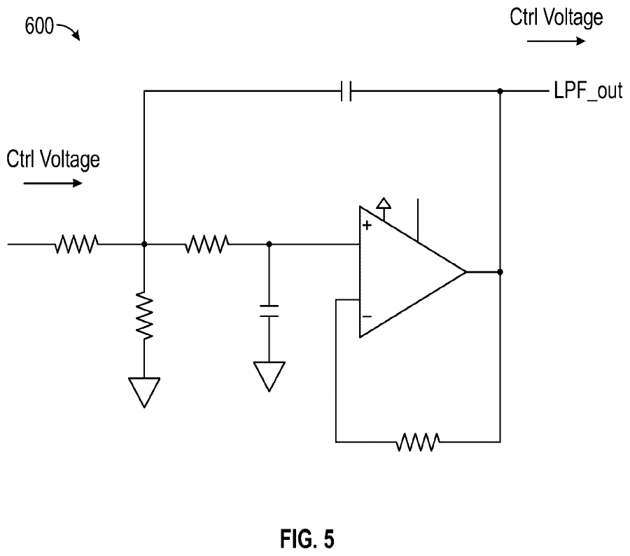

FIG. 3A shows a block diagram of a control loop 400 of the interrogation and detection system 10. The control loop 400 includes a Q factor control circuit 500, a controller 200, and a low pass filter 600 (FIG. 5). The Q factor control circuit 500 generally includes a sensor 420 and a voltage-controlled resistor 410 (FIG. 4). A voltage-controlled resistor 410 (VCR) is typically a three-terminal active device with one input port and two output ports. The input-port voltage controls the value of the resistor between the output ports. Voltage-controlled resistors are most often built with field-effect transistors (FETs), for example, a JFET and/or a MOSFET. The junction capacitance of the voltage-controlled resistor 410 may limit the speed at which the resistance can be adjusted as well as the real portion of the impedance that may be adjusted by the VCR. A voltage-controlled resistor 410 may include one or more devices in series.

The control loop 400 is configured to measure the current in series with the antenna 110 and adjust the channel impedance of one or more series voltage-controlled resistors 410 in order to maintain a constant current through the antenna 110.

The sensor 420 is configured to sense a signal, e.g., an electrical parameter such as current and/or voltage, and communicate the electrical parameter to the controller 200. The sensor 420 may include a root mean square (RMS) converter. For the measurement of an alternating current the signal is often converted into a direct current of equivalent value, the root mean square (RMS).

The controller 200 determines if the ratio of real impedance to reactive impedance is within a desired predetermined range of values (e.g., a window), based on the sensed signal. If the ratio is outside of the range of values, then an error current and/or voltage may be generated by the controller 200 and communicated to the low pass filter 600.

In aspects, the interrogation and detection system 10 may include a nested control loop.

FIG. 3B shows a block diagram of a nested control loop 400′ that may include portions of the control loop of FIG. 3A. The nested control loop 400′ generally includes a “fast loop” that corresponds to the control loop of FIG. 3A and a “slow loop.” The “slow loop” includes a programmable high voltage direct current (HVDC) supply 430 and an amplifier 440. The nested control loop 400′ is configured to servo the voltage amplitude of the antenna source RF signal to minimize the thermal load on the VCR 410 as well as regulating the Q-factor controller's bias point, enabling maximum Q-factor control over dynamic range. During transient events, the thermal load on the VCR 410 may exceed the rated voltage amplitude. The “slow loop” is configured to reduce the heat dissipated by reducing the amplifier 440 output power in scenarios where antenna Q-factor shifts higher during use.

Referring to FIG. 4 the Q factor control circuit 500 of the control loop of FIG. 3A is shown. The Q factor control circuit 500 typically includes a voltage-controlled resistor 502, an RF matching network 506, and a sensing resistor 504. The sensing resistor is typically a relatively small value resistor, such as about 0.5 ohms, and is configured for sensing an antenna current (Iₐₙₜₑₙₙₐ) by communicating a voltage drop (VCR+ and/or VCR−) across sensing resistor 504 to the sensor 420. The Q factor control circuit 500 may include other filtering components such as inductor and/or capacitors to process control voltages.

Typically, the body or tissue of a patient 18 presents an electrical impedance to the antenna 110, as the antenna 110 moves closer to the patient 18 (FIG. 1), the impedance rises and vice-versa. The total impedance seen by the output of the signal generator 300 (FIG. 1) is the sum of the antenna impedance and the impedance of one or more voltage-controlled resistors 502 in the Q factor control circuit 500. An RF matching network 506 may be used to match the antenna impedance to the impedance of the signal generator 300. By modulating the impedance of the voltage-controlled resistors 502 the total impedance seen by the signal generator 300 (FIG. 1) is more or less a constant.

For maximum power transfer, the total impedance seen by the signal generator 300 (FIG. 3A) should be a relatively constant impedance. This constant impedance enables any RF matching network 506 to be either drastically minimized or eliminated. Additionally, the transmit power of the signal generator 300 can be constant (or at least maintained within a small range of values) which has the benefit of helping with regulatory agency requirements.

The control loop may have a loop bandwidth of about 1/100 ms.

FIG. 5 is a schematic of an exemplary loop filter of the control loop of FIG. 3A. The low pass filter 600 (FIG. 5) is configured to integrate the error current to generate a Q factor control voltage that is communicated to the Q factor control circuit 500 (FIG. 4). The loop filter 600 may be an active or a passive filter. An active loop filter includes an active device such as an operational amplifier and typically has a high input impedance. The loop filter 600 may include one or more poles.

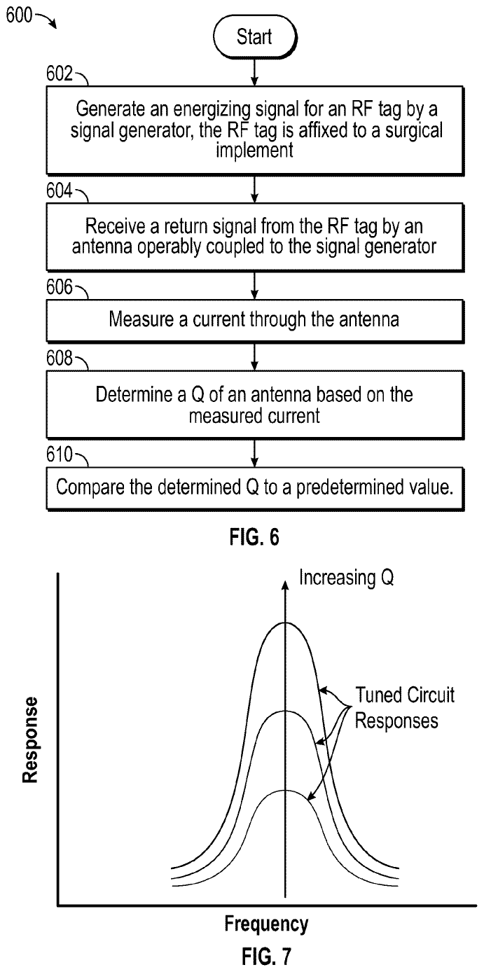

Referring now to FIG. 6, there is shown an operation for detection of surgical implements within a body of a patient. In various embodiments, the operation of FIG. 6 can be performed by an interrogation and detection system 10 described above herein (FIG. 1). In various embodiments, the operation of FIG. 6 can be performed by another type of system and/or during another type of procedure. The following description will refer to an interrogation and detection system, but it will be understood that such description is exemplary and does not limit the scope and applicability of the disclosure to other systems and procedures.

Initially, at step 602, the signal generator 300 generates an energizing signal for an RF tag 100 (FIG. 1). The RF tag 100 may be affixed to a surgical implement. As the antenna 110 is waved over a patient 18 (FIG. 1), the dielectric constant of the tissue may change the Q factor and/or tuning of the antenna 110.

Next, at step 604, the controller 200 receives a return signal from the RF tag by an antenna 110 operably coupled to the signal generator 300.

Next, at step 606, the controller 200 measures a current through the antenna 110. The current may be measured by an RMS converter, and/or sensor 420 (FIG. 3A). For example, a current of about 425 mA RMS (600 mA p-p) may be measured. The current may be measured across a series resistor such as sensing resistor 504. The measured antenna current (Iₐₙₜₑₙₙₐ) (FIG. 4) would be communicated to the controller 200. In aspects, RMS currents may fall between approximately 0.7 A-2 A for maximized reading distance (tag dependent) and regulatory compliance, although other currents are contemplated.

Next, at step 608, the controller 200 determines a Q factor of the antenna 110 based on the measured current. The Q factor of the antenna 110 is the ratio of the reactive portion of the impedance to the real portion of the impedance (Q=X_{L}/R_{L}). For example, the ratio may be about 30:1. Although 30:1 is used as an example, other ratios are contemplated. An antenna may have for example, an inductance of about 1.68 uH, a real impedance R_{L} of about 5 ohms, and an operating frequency of about 13.56 MHz, which would have an impedance with a reactive portion X_{L} of about 143 ohms. The Q factor (or determined Q) would be approximately 29 (Q=143 ohms/5 ohms).

Next, at step 620, the controller 200 compares the determined Q to a predetermined value of Q. For example, the predetermined value may be Q=35. The predetermined value of 35 may be compared to the determined (or measured) value of 29. The controller 200 may adjust the Q factor of the antenna 110 based on the comparison. For example, a voltage-controlled resistor 410 (FIG. 3A) may be adjusted by the controller 200 to either increase or decrease the resistance in response to the detected impedance of the antenna 110, by changing a tuning voltage of the voltage-controlled resistor 410. The change in resistance would increase or decrease the Q of the antenna 110. For example, the voltage-controlled resistor's 410 value may be decreased to about 4 ohms, which would yield a Q factor of about 35.

In aspects, the controller 200 may detect an impedance of the antenna based on the return signal. The impedance may include a real impedance and a reactive impedance. The impedance may be detected based on a current through the antenna 110.

Setting the ratio to the predetermined value may be performed by adjusting either a real element and/or a reactive element of the antenna. For example, a voltage-controlled resistor 410 may be adjusted by the controller 200 to either increase or decrease the resistance in response to the detected impedance of the antenna 110, by changing a tuning voltage of the voltage-controlled resistor 410. The adjusting may be performed by changing the current through the antenna 110 (FIG. 1).

In aspects, the controller 200 may determine a ratio of real impedance to reactive impedance based on the detected impedance. The controller 200 may set the ratio to be a predetermined constant value (e.g., about +/−0.1 ohm) based on the determined ratio of real impedance to reactive impedance.

The controller 200 may set the ratio to be within a predetermined value range based on the determined ratio of real impedance to reactive impedance. The ratio is a unitless number (ohms/ohm). For example, the controller 200 may maintain a value (ratio) of 10-20 ohms per ohm depending on the final application. An uncompensated (no Q factor control) resonant inductive antenna may have a Q-factor shift from >30 to <10 resulting in a field strength reduction of upwards of 70% in the presence of a tissue load when compared to free-space. The Q-factor control circuit 500 intentionally reduces the free-space field strength (degrades the Q-factor) in order to get “room to work with” (give Q factor back) when the antenna is loaded by tissue. The “giving Q-factor back” operation may be performed very quickly and with low computational overhead with the disclosed technology, which is one of the features that makes the Q-factor control circuit 500 so effective. Alternatively, the taking Q-factor away (like in a rapid transition from patient loaded to free-space) is also very quick, allowing for compliance with radio regulations in the free space case while maximizing field strength (still compliant) in the loaded case.

Referring to FIG. 7, a graph of Q factor vs. frequency as a real component of a tuning circuit is increased is shown. As the real element of the antenna is increased, the Q factor is a lower value. Although described as an element of an RF tag based system, it is contemplated that the Q-factor control concepts described herein extends to any inductive (coil) antenna-based system where field strength correlates directly with energizing/detecting performance.

While aspects of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular aspects. Those skilled in the art will envision other modifications within the scope and spirit of the claims appended hereto.

What is claimed is:

1. An interrogation and detection system for detection of surgical implements within a body of a patient, the interrogation and detection system comprising:
an RF tag configured to transmit a return signal when energized;
a signal generator configured to generate an energizing signal for the RF tag;
an antenna operably coupled to the signal generator, the antenna configured to receive the return signal transmitted by the RF tag;
a processor; and
a memory, including instructions stored thereon, which when executed by the processor, cause the system to:
receive the return signal;
detect an impedance of the antenna based on the return signal, wherein the impedance includes a real impedance and a reactive impedance; and
determine a ratio of real impedance to reactive impedance based on the detected impedance.

2. The system of claim 1, wherein the instructions, when executed by the processor, further cause the system to set the ratio to be a predetermined constant value based on the determined ratio of real impedance to reactive impedance.

3. The system of claim 1, wherein the instructions, when executed by the processor, further cause the system to set the ratio to be within a predetermined value range based on the determined ratio of real impedance to reactive impedance.

4. The system of claim 3, wherein setting the ratio to the predetermined value is performed by adjusting at least one of a real or a reactive element of the antenna.

5. The system of claim 4, wherein the impedance is detected based on a current through the antenna.

6. The system of claim 5, wherein the adjusting is performed by changing the current through the antenna.

7. The system of claim 4, wherein the adjusting is performed by changing a tuning voltage to a voltage-controlled resistor.

8. The system of claim 5, wherein the current is measured by a root mean square (RMS) converter.

9. The system of claim 1, wherein the instructions, when executed by the processor, further cause the system to determine whether a return signal was received via the antenna from an RF tag that marks a surgical implement used in a procedure.

10. The system of claim 9, wherein the instructions, when executed by the processor, further cause the system to:
transmit information to a display configured to display information related to the RF tag.

11. An interrogation and detection system for detection of surgical implements within a body of a patient, the interrogation and detection system comprising:
an RF tag configured to transmit a return signal when energized;
a signal generator configured to generate an energizing signal for the RF tag;
an antenna operably coupled to the signal generator, the antenna configured to receive the return signal transmitted by the RF tag;
a processor; and
a memory, including instructions stored thereon, which when executed by the processor, cause the system to:
measure a current through the antenna;
determine an electrical damping factor (Q factor) of the antenna based on the measured current; and
compare the determined Q factor to a predetermined value.

12. The system of claim 11, wherein the instructions, when executed by the processor, further cause the system to adjust the Q factor of the antenna based on the comparison.

13. The system of claim 11, wherein the adjusting is performed by changing the current through the antenna.

14. The system of claim 11, wherein the adjusting is performed by changing a tuning voltage to a voltage-controlled resistor.

15. The system of claim 11, wherein the current is measured by an RMS converter.

16. A computer-implemented method for detection of surgical implements within a body of a patient, the method comprising:
generating, by a signal generator, an energizing signal for an RF tag, the RF tag affixed to a surgical implement;
receiving a return signal from the RF tag by an antenna operably coupled to the signal generator;
measuring a current through the antenna;
determining a Q factor of the antenna based on the measured current;
comparing the determined Q factor to a predetermined value; and
adjusting the Q factor of the antenna based on the comparison.

17. The computer-implemented method of claim 16, wherein the adjusting is performed by changing the current through the antenna.

18. The computer-implemented method of claim 17, further comprising:
determining whether a return signal was received from the RF tag that marks a surgical implement used in a procedure via the antenna.

19. The computer-implemented method of claim 17, wherein the current is measured by an RMS converter.

20. The computer-implemented method of claim 17, further comprising:
transmitting information to a display configured to display information related to the RF tag.
